(19) United States
(12) Patent Application Publication
(10) Pub. No.: US 2013/0344579 A1
(43) Pub. Date: Dec. 26, 2013

(54) IMAGING CHAMBER FOR SUPPORTING MULTIPLE INVESTIGATION OF CELLS AND TISSUES BY VARIOUS TECHNIQUES

(71) Applicants: Viktor Izápy, Budapest (HU); Õdön Wagner, Budaors (HU); Zsombor Lacza, Csopak (HU); Miklós Weszl, Budapest (HU)

(72) Inventors: Viktor Izápy, Budapest (HU); Õdön Wagner, Budaors (HU); Zsombor Lacza, Csopak (HU); Miklós Weszl, Budapest (HU)

(73) Assignee: LACERTA TECHNOLOGIES INC., WINSTON-SALEM, NC (US)

(21) Appl. No.: 13/978,195

(22) PCT Filed: Jan. 11, 2011
(86) PCT No.: PCT/HU11/00003

§ 371 Date: Sep. 16, 2013

(51) Int. Cl. C12Q 1/02

(52) U.S. Cl. CPC C12Q 1/02
USPC 435/288.3

(57) ABSTRACT

The imaging chamber according to the invention comprises a housing with a lower part and an upper part, wherein said lower part is a dish (1) and said upper part is a lid (2) provided with a skirt (9) surrounding the upper rim (10) of the dish (1), the bottom of the dish (1) is provided with a cover slip (12) to be used in confocal microscopy and at least a part of the lid (2) is transparent wherein the lid (2) is provided with a breathing silicon insert (5). The silicon insert (5) comprises a circular ring part fixed to the inner surface of the lid (2) and a skirt (14) parallel with the upper rim (10) of the dish (1) and supported by the skirt (9) of the lid (2) and provided with an annular groove (15) tight fitted on the upper rim (10) of the dish (1); and the lid (2) is provided with openings (4) to allow breathing of the housing.

### OBJECT OF THE INVENTION

The present invention relates to an imaging chamber for expanding cells under standard cell culture conditions and/or for long-term investigation of cultured cells or tissues by various imaging techniques, such as light microscopy, confocal microscopy, micro-CT, and nanoSPECT/CT.

### BACKGROUND OF THE INVENTION

Over the years, many customized live-cell imaging chambers have been designed to meet specific requirements, and a wide range of commercial perfusion and imaging chambers are now available. These chambers include glass bottom Petri dishes, multi-well chambers mounted on microscope slides, heating stages with a variety of interchangeable perfusion adapters, and specialized chambers with conductive coatings for tight control of temperature.

U.S. Pat. No. 4,839,046 (Joseph A. Chandler) discloses a bioreactor chamber which provides a fresh supply of oxygenated fluid nutrient medium into the chamber without excessive turbulent action in the cell culture. This chamber, however, is not applicable with any imaging instrument, due to its shape.

U.S. Pat. No. 5,6655,99 (Will Minuth) relates to a multipart chamber for cultivating cells and for observing long-term cell cultures by microscopy. It discloses a closed system wherein tightness is provided by a fixing ring (Seeger-ring) and O-rings. The chamber allows the perfusion of cells and a glass bottom enables their investigation in visual light.

WO/2004/091509 (Akers et al) discloses a cell culturing chamber, however, it is not designed as an imaging device. It can be used as a single chamber, or it can be integrated into a system with two interlinked chambers, in which cells, tissues and other biologicals are cultured. The two culture compartments are in fluid communication with each other and each culture compartment is transversed by a filter that prevents the exit of the cells from the culture compartment.

U.S. Pat. No. 5,162,204 (Harumi Matsuzaki et al) suggests a complete system for cell culturing and diagnosis using transmission light microscopy and image processing. It is supplied with a control system to control the culture conditions of a culturing cistern on the basis of a result of the diagnosis, and a culture medium regenerating system to remove waste contents in a culture medium. In the apparatus, the culture system and diagnosis system are cut off from the outside of the apparatus system. Therefore, no infection of miscellaneous microorganism, etc., occurs, nor does consumption of cells and products occur.

The company Greiner Bio-One developed a cell culturing chamber with a glass bottom in order to support the microscopic investigation of cells. By its appearance this technical solution is the closest to our invention, however, it does not allow the long-term investigation of cells and tissues due to the lack of perfusion supply and sealing closure. Furthermore, it is not adaptable with diverse imaging instruments. The common characteristics of the known imaging chambers is their single applicability. Typically, those imaging chambers are adaptable with only one imaging instrument, such as light or confocal microscope for single purpose.

Therefor, object of the present invention is to provide an imaging chamber which may be used with various imaging instruments providing long-term, multiple investigational opportunities of the same biological sample (cells or tissues).

### SHORT DESCRIPTION OF THE INVENTION

The main features of the imaging chamber according to the invention are recalled in claim 1. Further features may be found in claims 2 to 13.

The chamber comprises two main parts: a dish and a perforated lid. The dish is preferably made of a transparent polymer and has a cover slip fused to the base. A perforated lid is preferably made of a transparent polymer, which permits the visual light allowing the investigation of cells and tissues by light microscopy. The inner surface of the perforated lid is sealed by a silicon insert, which allows gas exchange. The lid is tight fitted to the edge of the dish and keeps the contagious agents out of the chamber. The lid further is provided with inlets and outlets allowing the perfusion of cells or tissues without opening of the chamber.

The closed system according to the invention enables the long-term investigation of cells and tissues in the ambient atmosphere without contamination.

The imaging chamber has a unique silicon closure ring insert fixed within the perforated lid, providing tight fitting between the lid and the dish. It inhibits leaking but allows gas exchange at the same time through openings of the lid. Both the lid and dish are preferably made of a water clean polymer, which allows the investigation of cells and tissues both by confocal microscopy and by light microscopy. In order to support the confocal microscopic application, the base of the polymer dish is provided with a cover slip. The imaging chamber according to the invention does not contain any metallic parts, and therefor is can be used in Micro-CT and NanoSPECT/CT imaging applications as well.

The silicon ring insert consists of thick and thin parts. The thick part is responsible for the tight sealing and the mounting in the lid, whereas the thin parts provide ‘breathing membrane’ allowing gas exchange in the chamber. The ‘breathing membranes’ are right under the openings of the perforated lid.

According to another embodiment, the thin and the thick parts of the silicon insert are separate elements, wherein the thin “breathing membrane” may be a flat ring arranged between the lid and the thick part or the insert. A regular membrane filter of 0.22 μm pore-size may also applied for the gas exchange instead of a silicon film. Beside the gas exchange, the holes on the lid are used for the mounting of the silicon insert and allowing perfusion. The silicon ring can be fixed to the inner surface of the lid by several ways, for example, by connecting grooves and edges on different faces of the lid and the silicone, or the silicon ring can be glued into the lid.

There may be a little shoulder on the outer diameter of the bottom of the dish in order to protect the glass cover slip from scratch. On the top of the lid seats may be arranged, allowing the piling of several chambers.

### BRIEF DESCRIPTION OF THE DRAWINGS

The invention will now be described by way of non limiting examples, with reference to the enclosed drawings, wherein:

FIG. 1. is an axonometric view of a preferred embodiment of the imaging chamber according to the invention;

FIG. 2. is a cross-section of the imaging chamber shown in FIG. 1.; and

FIG. 3 is an exploded view of the lid and a two part insert of another embodiment of the invention.

### DETAILED DESCRIPTION OF THE EXEMPLARY EMBODIMENTS

In the following description, various specific details are illustrated to provide a thorough understanding of the embodiments. However, the embodiments can be practiced without one or more of the specific details, still being within the scope claimed in the attached claims.

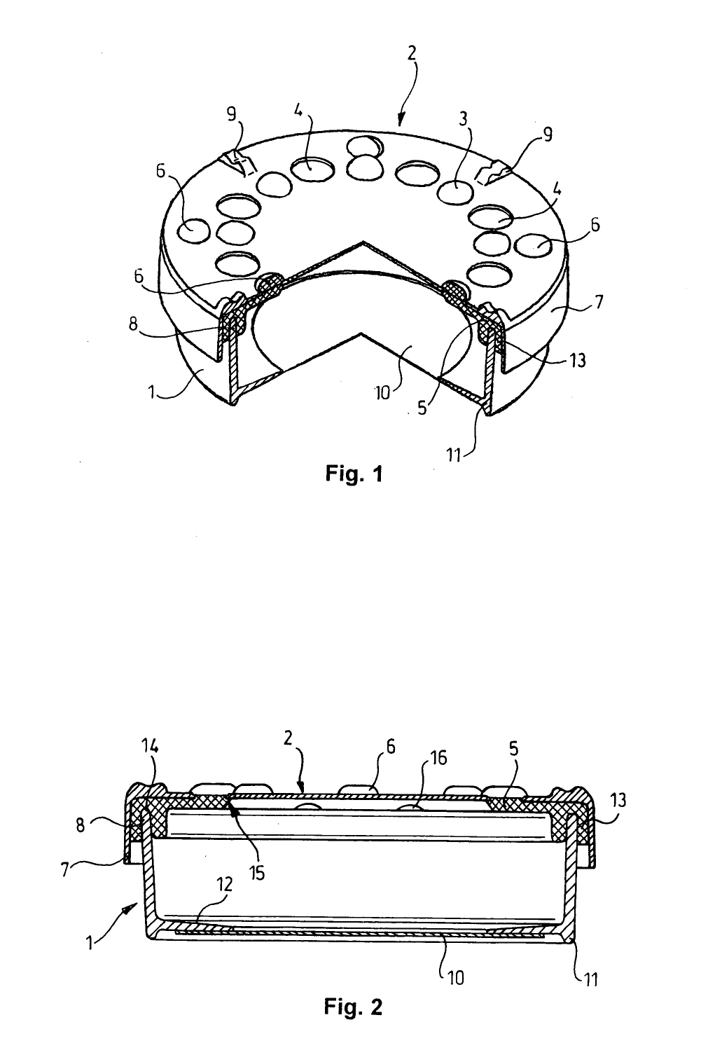

FIGS. 1 and 2 show the two main housing parts: a dish 1 and a lid 2. The upper housing part, the lid 2 is made of a fully transparent (water clear) polymer in order to allow the investigation by transmission light microscopy. There are openings 3, 4 of different sizes arranged in the lid 2. The smaller openings 3 fasten a breathing annular silicon insert 5 arranged within the lid 2 by extensions 6 inserted into this openings. The larger openings 4 enable breathing through insert 5 and provide connection surfaces for perfusion tubes. Around its perimeter, the lid 2 is provided with a skirt 7 surrounding the upper rim 8 of the dish 1. Further, there are seats 9 for supporting another imaging chamber's bottom edge (shoulder) when chambers are piled on each other.

The lower housing part, dish 1 is made of a polymer and has a glass bottom (cover slip) 10. The dish has a protruding annular rib (shoulder) 11 around its outer diameter, which lifts the glass bottom in order to prevent scratching of the cover slip 10 and enables to pile the chamber on the top of another chamber. The cover slip 10 is preferably fused to the bottom plane surface of the dish. The polymer bottom surface of the dish has a slight inclination 12 towards the middle of the chamber.

The silicon insert 5 has a peripheral skirt 13 parallel with the upper rim 8 of the dish 1, supported by the skirt 7 of the lid 2 and provided with an annular groove 14 tight fitted on the upper rim 8 of the dish 1. The groove 14 is narrower by a few hundredths of millimeters than the edge 8 of the dish 1, in order to ensure the tight fitting and sealing 25 between the dish 1 and the lid 2.

The inner peripheral edge of the silicon insert 5 has a chamfer 15 and bends towards the lid to guarantee the proper sealing between this parts. There are narrow sections 16 of the silicon insert 5 below the larger openings 4 of the lid 2 to enhance gas exchange between the chamber and the surrounding atmosphere (breathing).

The extensions 6 may be of mushroom shape (FIGS. 1 and 2), having a bit shorter neck than the thickness of the lid 2, in order to ensure proper sealing between the lid 2 and the silicon insert 5. The extensions 6 can have, however, any other shape providing a reliable connection between the lid and the insert.

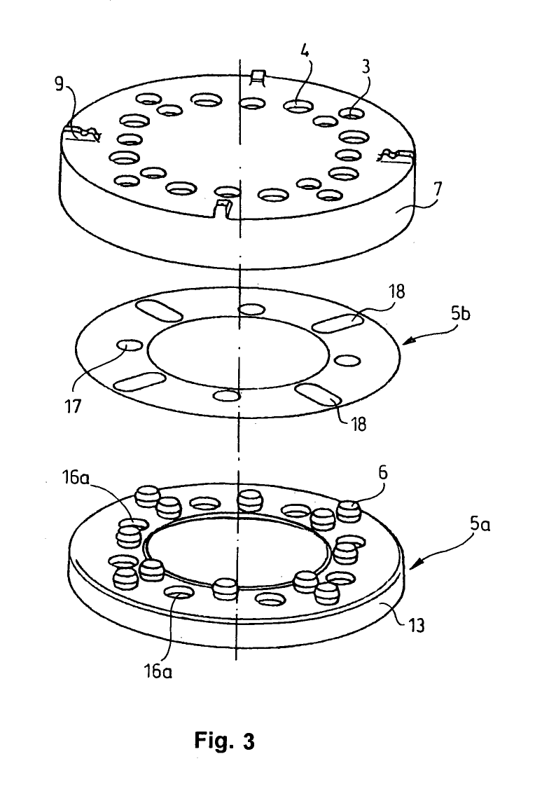

According to another embodiment of the invention shown in FIG. 3, the insert 5 consists of a thick lower part 5a and a separate, thin upper part 5b. There are no narrow sections 16 in the thick upper part 5a below the larger openings 4 of the lid 2, but openings 16a are provided in correspondence with the larger openings 4 of the lid. Breathing is ensured by openings 17, 18 arranged in the thin upper part 5b in correspondence with and the openings 3 of the lid 2 and the extensions 6 of the lower part 5a of the insert 5. The extensions 6 here have a pineapple cone shape and provide connection between the insert and the lid.

Taking into consideration the various possible manufacturing processes the latest solution may provide a better and cost-effective way of production than the first one.

The imaging chamber according to the invention can be advantageously used for expanding cells under standard cell culture conditions and/or for long-term investigation of cultured cells or tissues by various imaging techniques, such as light microscopy, confocal microscopy, micro-CT, and nanoSPECT/CT. As the lid is provided with openings allowing the perfusion of cells or tissues without opening of the chamber, it ensures multiple investigational opportunities of the same biological sample (cells or tissues).

The description and figures illustrate non limiting embodiments of the present invention. However, the invention compasses many other variants, which are all within the scope claimed in the attached claims. The silicon insert e.g. may be fixed to the inner surface of the lid by resilient extensions inserted in corresponding channels on the inner surface of the lid or may simply be glued to the inner surface of the lid. The extensions on the insert may also have many different shapes further to the mushroom or pineapple cone shape shown in the examples, and the arrangement of the openings and shape of the other elements may be different, without departing from the principle of the inventive concept defined by the annexed claims.

What we claim is:

1. Imaging chamber for treating and/or investigating cells, comprising a housing with a lower part and an upper part, wherein
said lower part is a dish (1) and said upper part is a lid (2) provided with a skirt (9) surrounding the upper rim (10) of the dish (1),
the bottom of the dish (1) is provided with a cover slip (12) to be used in confocal microscopy and
at least a part of the lid (2) is transparent
characterized in that
the lid (2) is provided with a breathing silicon insert (5);
said silicon insert (5) comprises a circular ring part fixed to the inner surface of the lid (2) and a skirt (14) parallel with the upper rim (10) of the dish (1) and supported by the skirt (9) of the lid (2) and provided with an annular groove (15) tight fitted on the upper rim (10) of the dish (1); and
the lid (2) is provided with openings (4) to allow breathing of the housing.

2. The imaging chamber according to claim 1, wherein said silicon insert (5) is fixed to the inner surface of the lid (2) by resilient extensions (6) inserted in corresponding openings (3) of the lid (2).

3. The imaging chamber according to claim 1, wherein said silicon insert (5) is fixed to the inner surface of the lid (2) by resilient extensions inserted in corresponding channels on the inner surface of the lid (2).

4. The imaging chamber according to claim 1, wherein silicon insert (5) is glued to the inner surface of the lid (2).

5. The imaging chamber according to claim 1, wherein the silicon insert (5) is provided with narrow sections (20) below the openings (3).

6. The imaging chamber according to claim 1, wherein the openings (3) for fixing the silicon insert (5) are smaller, then the openings (4) for breathing.

7. The imaging chamber according to claim 1, wherein the extensions (6) of the silicon insert (5) are of mushroom or pineapple cone shape.

8. The imaging chamber according to claim 1, wherein there are seats (11) on the top of the lid (2) for supporting another imaging chamber's bottom.

9. The imaging chamber according to claim 1, wherein the dish (1) has a protruding annular rib (13) around its outer diameter.

10. The imaging chamber according to claim 1, wherein the inner peripheral edge of the silicon insert (5) is provided with a chamfer (19) and bends towards the lid (2).

11. The imaging chamber according to claim 1, wherein the dish (1) is made of a polymer.

12. The imaging chamber according to claim 11, wherein the cover slip (12) is fused to the bottom plane surface of the dish (1).

13. The imaging chamber according to claim 1, wherein the silicon insert (5) consists of a thick lower part (5a) without narrowed parts (16) and a separate, thin upper part (5b) provided with openings (17, 18) arranged in correspondence with the openings 3 of the lid.
